(19) United States
(12) Patent Application Publication
(10) Pub. No.: US 2005/0215787 A1
(43) Pub. Date: Sep. 29, 2005

(54) AN IMPROVED PROCESS FOR THE PREPARATION OF PURINES

(76) Inventors: Ramesh Anna Joshi, Maharashtra (IN); Rohini Ramesh Joshi, Maharashtra (IN); Pratap Subhashrao Patil, Maharashtra (IN); Vijay Naryan Wakchaure, Maharashtra (IN); Mukund Keshav Gurjar, Maharashtra (IN)

Correspondence Address: CHRISTIE, PARKER & HALE, LLP, PO BOX 7068, PASADENA, CA, 91109-7068, US

(21) Appl. No.: 10/809,967

(22) Filed: Mar. 26, 2004

(51) Int. Cl. C07D 473/10

(52) U.S. Cl. 544/276; 544/277

(57) ABSTRACT

The present invention relates to a process for the preparation of Purines. More particularly it relates to the preparation of Purines of formula (1) 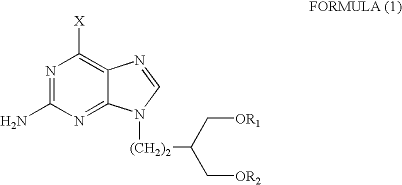
wherein X is hydrogen, thioaryl; R₁ and R₂ are hydrogen or acetyl.

### FIELD OF INVENTION

The present invention relates to a process for the preparation of Purines. More particularly it relates to the preparation of Purines of formula (1) 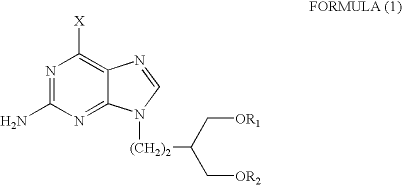
wherein X is hydrogen, thioaryl; R₁ and R₂ are hydrogen or acetyl

### BACKGROUND AND PRIOR ART REFERENCES

The 2-amino-6-thiosubstituted purines are useful intermediates in the preparation of nucleoside analogue antiviral agents such as Famciclovir of formula (6) Penciclovir of formula (7).

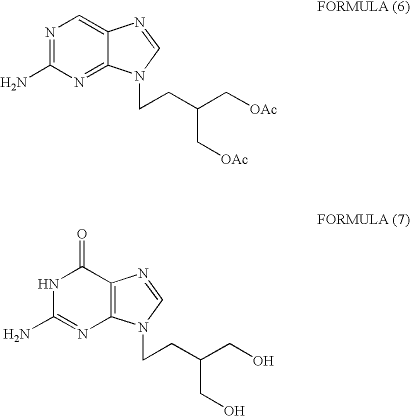

In the prior art the compound of formula (1) are prepared by various methods as shown hereinbelow.

The EP patent No. A0352953 describes the preparation of purine derivatives by the reaction of compound of formula (2) wherein X is benzythio, phenacyl methyl thio, with a side chain intermediate of formula (3) wherein R₁ and R₂ are acyl groups or hydroxy protecting groups and Q- is a leaving group.

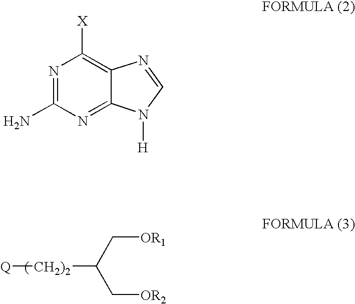

Another process disclosed in a PCT application No. WO95/28402 and Nucleosides and nucleotides 15(5), 981-994 (1996) involves method for the preparation of formula (1) by reacting 2-amino-6-chloropurine of formula (2, X=Cl) with the compound of formula (4) where in Q is leaving group and R is alkyl group.

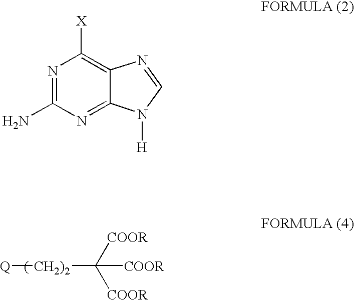

The above methods are disadvantageous as the alkylation of intermediate (2) results in a mixture of N-7 and N-9 alkylated products when reacted with side chain compound of formula (3) or (4) resulting in low yields of the desired N-9 product due to chromatographic separation of mixtures.

The disadvantages with the process for the preparation of thio derivative [formula (2)] wherein X— is thio aryl is that the method requires use of reagents like trifluoroacetic anhydride, pyridine, cryogenic temperature, hydrogen peroxide making it difficult to scale up.

It is observed that if the preparation of compound of formula (1) is carried out via an intermediate of formula (5) formed by reaction of compound (2) with compound (4) the disadvantages of the prior art are eliminated as via this route N-7 alkylated isomer is formed in very low percentage which remains in solvent phase during isolation and the desired N-9 alkylated isomer is formed in higher percentage.

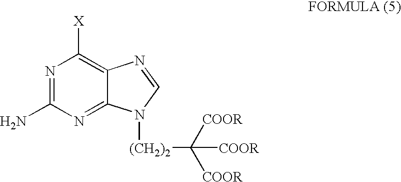

### OBJECTIVE OF INVENTION

The main object of the present invention therefore is to provide a process for the preparation of purines of formula (1) in high yields.

Another object is to provide a process for the preparation of these compounds via formation of new compound of formula (5) so as to eliminate the disadvantages of the prior art methods.

Still another object is to provide a process wherein the use of certain reagents like trifluoroacetic anhydride, pyridine, cryogenic temperature, hydrogen peroxide is avoided making it easy to scale up for the preparation of compound of formula (2).

Yet another object is to provide a process in which the separation of N-9 and N-7 alkylated purine derivatives requires no column chromatography and N-9 alkylated derivative can be isolated directly in high yield.

### SUMMARY OF INVENTION

The present invention relates to a process for the preparation of Purines. More particularly it relates to the preparation of Purines of formula (1) 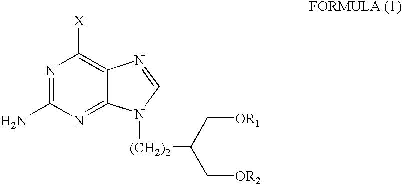
wherein X is hydrogen, thioaryl; R₁ and R₂ are hydrogen or acetyl

### DETAILED DESCRIPTION OF INVENTION

Accordingly the present invention provides a process for the preparation of purines of general formula (1) 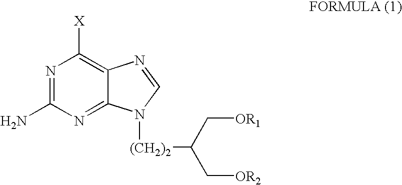
wherein, wherein X is 4-methylphenylthio, 4-chlorophenylthio and R1 and R2 are Hydrogen or acetyl group.

Which comprises;
(a) reacting an aminopurine derivative of formula (2), 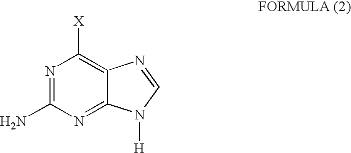
wherein X is 4-methylphenylthio, 4-chlorophenylthio with a triester of formula (4) 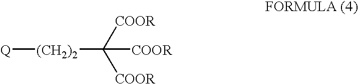
wherein Q is leaving group and R is C₁₋₆ alkyl preferably methyl or ethyl group, in presence of an organic solvent under constant agitation at about 50° C. for a period of 2 to 5 hrs. to obtain an intermediate derivative of formula (5) 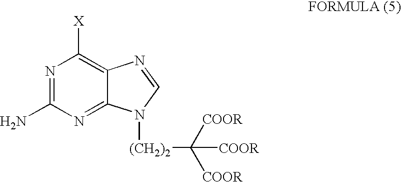
wherein X is 4-methylphenylthio, 4-chlorophenylthio and R is C₁₋₆ alkyl preferably methyl or ethyl group;
(b) cooling the reaction mixture to a temperature at about 15° C. to obtain the solid intermediate derivative of formula (5);
(c) treating the compound of formula (5) with an alkoxide base in an alcoholic solvent at ambient temperature to obtain a diester;
(d) reducing and acylating the diester in situ to obtain the intermediate compound of formula (6), and 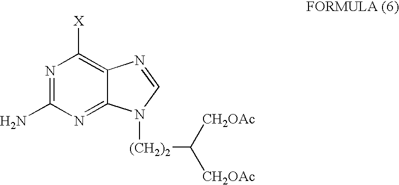
(e) desulfurising the intermediate of formula (6) with Raney nickel to obtain the compound of formula (1). 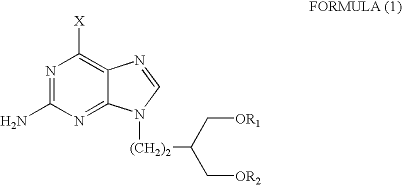
   which comprise reacting an aminopurine derivative of formula (2) 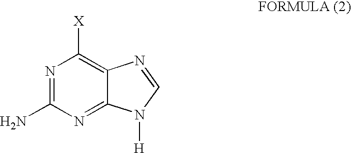
   wherein X is 4-methylphenylthio, 4-chlorophenylthio with a triester of formula (4) 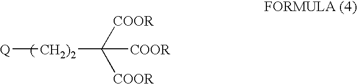
   wherein Q is leaving group and R is C₁₋₆ alkyl preferably methyl or ethyl group, in presence of an organic solvent under constant agitation at 50° C. for a period of 2 to 5 hrs. to obtain an intermediate derivative of formula (5).
   wherein X and R are as in formula (2) and (4) cooling the reaction mixture to a temperature at ˜15° C. and isolating the solid intermediate derivative of formula (5) 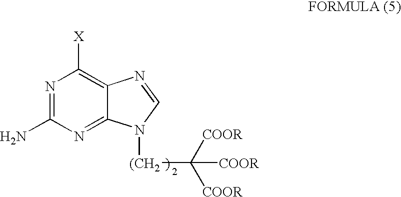
   by conventional methods treating the compound of formula (5) with Sodium methoxide base in an alcoholic solvent at ambient temperature to obtain a diester, reducing and acylating the diol in situ by conventional methods to obtain the intermediate compound of formula (6). 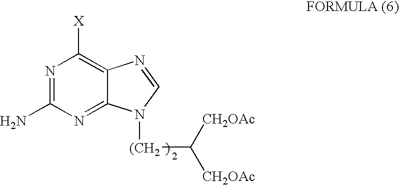
   desulfurising the intermediate of formula (6) with Raney nickel to obtain the compound of formula (1)

In one of the embodiments of the present invention the organic solvent used for preparing compound (2) and washing of compound (4) may be alcohol such as methyl and ethyl alcohol.

In another embodiment the alkoxide base may be alkoxide base of alkali metals preferably sodium methoxide.

In another embodiment of the present invention preparation of 6-thioderivative may be carried out by reacting 2-Amino-6-chloropurine with arylthiol in an alcoholic solvent and an organic base over a temperature range of 0° C. to boiling point of solvent preferably 25-30° C.

In still another embodiment the organic bases used for the preparation of 2-Amino-6-chloropurine are triethylamine, ethyldiisopropylamine, DBU in an alcoholic solvents such as methanol, ethanol, isopropanol.

In a feature of the present invention the acylation of the diol of compound (5) is done by conventional methods by using acetic anhydride as acylating agent. In yet another feature reaction for the preparation of intermediate of formula (5) may be carried out in an organic solvent for example DMF, DMSO, acetonitrile, preferably DMSO in the presence of inorganic or organic base over a temperature range from 0° to the boiling point of the solvent usually 60-70° C. Examples of inorganic bases include alkali metal hydrides, alkalimetal carbonates preferably potassium carbonate. Suitable organic bases are DBU (diazabicycloundecane), tetramethyl guanidine. Suitable examples of optional substituents in the phenyl group X include one or two groups selected from C₁₋₄ alkyl, halo. Halo includes iodo, bromo, chloro, fluoro, alkyl group include those containing methyl, ethyl and isopropyl. X is preferably 4-methyl or 4-chlorophenylthio, more preferably 4-methylphenylthio.

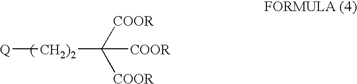

In still another feature intermediates of formula (4) are prepared by analogous methods. The compound of formula (4) wherein Q is bromo, R is ethyl may be prepared from triethylmethantricarboxylate according to the procedure described by H. Rapopart et. al. J. Org. Chem. 44, 3492 (1979).

The process of the present invention is described herein below with reference to examples which are illustrative only and should not be construed to limit the scope of the present invention in any manner.

HPLC analysis was carried out using column Rp C₁₈ Lichrocart 12.5 cm, wavelength 254 nm, flow 1 ml/min., system A (30:70 acetonitrile—buffer of pH—6.9) or B (15:85 acetonitrile—buffer pH—6.9). TLC analysis was carried out using Merk silicagel 60 F₂₅₄ plates in system A (1:19 methanol—chloroform v/v) and system B (1:49 methanol—chloroform v/v).

### EXAMPLE-1

### This example illustrates the preparation of 2-Amino-6-[(4-Methylphenyl)thio]purine. Formula (2) X=4-methyl phenyl thio

To the mixture of 2-Amino-6-Chloropurine (10 g, 1 Eq.), methanol, (200 ml.) and triethylamine (30 ml.; 1.3 Eq.) taken in round bottom flask p-thiocresol (47.5 g, 2.6 Eq.) was added in portions after regular intervals and the reaction mixture left at room temperature with stirring for 21 hrs.

Obtained solid filtered off, washed with pet ether and dried to get 13.6 g white solid.

Yield: 90%; mp: 235° C.; HPLC purity 98.1%, Rₜ 3.7, system-A;

¹H NMR [(CD₃)₂SO] δ 6.27 (brs, 2H, NH₂), 7.35 (d, 2H, Ar), 7.46 (d, 2H, Ar), 7.98 (s, 1H, —N—CH═N), 1.92 (s, 3H, CH₃—Ar) 12.63 (brs,1H, —NH—);

¹³C NMR [(CD₃)₂SO] δ 123.2, 127.2, 129.1, 133.8, 139.8, 152.7, 156.9, 159.7

### EXAMPLE-2

### This example illustrates the preparation of 2-Amino-6-[(4-chlorophenyl)thio]purine. Formula (2) X=4-chlorophenyl thio

Procedure and quantities for the reaction of 2-amino-4-chloropurine (10 g) with 4-chlorophenylthiol are in same equivalent as in example-1.

Yield: 86.58%; mp: 125° C.; HPLC purity 96%, Rₜ 5.3, system-A;

¹H NMR [(CD₃)₂SO] δ 6.27 (brs, 2H, NH₂), 7.50 (d, 2H, Ar), 7.63 (d, 2H, Ar), 7.98 (s, 1H, N—CH═N), (brs,1H, —NH—);

¹³C NMR [(CD₃)₂SO] δ 123.2, 127.2, 129.1, 133.8, 139.8, 152.7, 156.9, 159.7

### EXAMPLE-3

### This example illustrates the preparation of 2-[2-(2-Amino-6-p-tolylthio-purine-9-yl)ethyl]-2-ethoxycarbonylmalonic acid diethyl ester: Formula (5)

A mixture of 2-Amino-6-[(4-methylphenyl)thio]purine (15 g, 1 Eq.), K₂CO₃ (16.05 g, 2 Eq,), triethyl 3-bromopropane-1,1,1-tricarboxylate (23.7 g, 1.2 Eq,) and dry DMSO (8 ml) was stirred together at 50° C. After 4 hrs. water (10 ml) was added, stirred for 10 min. and extracted with DCM. The combined organic extract were washed with brine, dried over sodium sulphate and on concentration under vaccum was obtained 24.6 gm of the product.

Yield: 82%; HPLC purity 94%, Rₜ.19.83, system-A;

¹H NMR [CDCl₃] δ 7.98 (s, 1H, N—CH═N), 7.63 (d, 2H, Ar), 7.50 (d, 2H, Ar), 4.8 (brs, 2H, NH₂), 4.25 (m, 2H, —NCH₂—+—CH₂CH₃), 1.29 (s, 1H, Ar—CH₃), 2.65 (t, 2H, —CH₂—C—), 1.25 (t, 9H, —CH₃),

### EXAMPLE-4

### This example illustrates the preparation of 2-Amino-6-[(4-methylphenyl)thiol]-9-[methyl 2-carbomethoxybutanoate-4-yl]-9H purine:

To a cooled (20° C.) solution of triester (15 g, 1 Eq,) in methanol (350 ml), solution of sodium methoxide (2.3 g, 1.5 Eq,) in methanol (150 ml) was added with stirring at 20° C., stirring was continued for 4 hrs at same temperature. The reaction mixture was cooled to 15° C. and held at this temperature for 30 min. (till the complete product was precipitated). Product was filtered off washed with methanol (15 ml) and dried at 40° C. to yield 9.1 g of title compound

Yield: 75%; mp: 135° C.; HPLC: purity—98%, Rₜ.—15.65, system-A;

¹H NMR [CDCl₃] δ 7.65(s, 1H, —NCH═N—), 7.5 (d, 2H, Ar), 7.2 (d, 2H, Ar), 4.86(brs, 2H, NH2), 4.16(t, 2H, —CH₂—N—), 3.73(s, 3H, —OCH₃), 3.36 (t, 1H, —CH—COOMe), 2.45 (t, 2H, —CH₂—C—), 2.39 (s, 3H, CH₃—Ar)

### EXAMPLE-5

### This example illustrates the preparation of 2-Amino-6-[(4-methylphenyl)thio]-9-[4-hydroxymethyl)butyl]-9H purine

A mixture of 2-Amino-6-[(4-methylphenyl)thio]-9-[methyl 2-carbomethoxy butanoate-4-yl]-9H purine (10 g, 1 Eq,), sodium borohydride (3.5 g, 3.7 Eq,) and dry DCM (50 ml) were stirred at 20° C. Methanol (25 ml) was added drop wise over a period of 2 hrs. at 20° C. Then reaction mixture was left to stir for further 6 hrs. at r.t.

Water (50 ml) was added, followed by drop wise addition of conc. HCl to maintain pH 6.7 to 7.0 keeping the reaction temperature 20° C. Methylene chloride and methanol were removed under vacuum until a reaction volume (25 ml) was obtained. The reaction mixture was cooled to 5° C. and stirred at this temperature for 30 min. Precipitated product was filtered off and washed with pet-ether to get 7.8 g dry solid.

Yield: 90.5%, mp: 94° C.; HPLC: 94% purity, Rₜ 3.108, system-A;

¹H NMR [CDCl₃] δ 7.98 (s, 1H, N—CH═N), 7.63 (d, 2H, Ar), 7.50 (d, 2H, Ar), 6.4 (brs, 2H, —OH) 4.8 (brs, 2H, NH₂), 4.25 (m, 2H, —NCH₂—+—CH₂CH₃), 3.6 (d, 4H, —CH2—OH) 2.65 (t, 2H, —CH₂—C—), 1.29 (s, 1H, Ar—CH₃),

### EXAMPLE-6

### This example illustrates the preparation of 9-[4-Acetoxy-3-(acetoxymethyl) butyl] 2-amino-6-[(4-methylphenyl)sulfanyl]-9H purine Formula (1)

To a mixture of 2-Amino-6-[(4-methylphenyl)sulfanyl]-9-[(4-hydroxymethyl)butyl]-9H purine (10 g, 1 Eq.), NEt₃ (2.25 g, 0.8 Eq,) and 4-dimethylaminopyridine (catalytic) in DCM (350 ml), acetic anhydride (16.1 g, 0.568 Eq,) was added drop wise over 20 to 30 min. at such rate to control the reflux. The reaction mixture was heated under reflux for further 7 hrs.

The reaction mixture was cooled to 20° C. and neutralized with 20% NaOH solution to pH 6.4-6.5. The DCM layer was separated and extracted with DCM (3 times with 75 ml). The combined DCM layer evaporated and syrupy product used without purification for further reaction.

Yield: 71% HPLC 90% purity, Rₜ 18.267, system-A;

¹H NMR [CDCl₃] δ 7.98 (s, 1H, N—CH═N), 7.63 (d, 2H, Ar), 7.50 (d, 2H, Ar), 6.4 (brs, 2H, —OH) 4.8 (brs, 2H, NH₂), 4.25 (m, 2H, —NCH₂—+—CH₂CH₃), 3.6 (d, 4H, —CH2—OH) 2.65 (t, 2H, —CH₂—C—), 2.00(6H,s), 1.29 (s, 1H, Ar—CH₃),

### EXAMPLE-7

### This example illustrates the preparation of 9-[4-Acetoxy-3-(acetoxymethyl)butyl]-2-amino-9H purine: Formula (6)

9-[4-Acetoxy-3-(acetoxymethyl)butyl]-2-amino-6-[(4-methylphenyl)sulphenyl]-9H purine (7 g, 0.001 Mole), Raney-Nickel slurry (31.7 g) and ethanol-water (1:1 v/v; 200 ml.) were stirred together and heated under gentle reflux. After 3 hrs. the product was filtered through celite and the filtrate was evaporated under reduced pressure. Crude solid was purifed by flash chromatography on neutral alumina to obtain 3.8 g of final product.

Yield: 75%; HPLC purity 99%, Rₜ 6.567, system-B;

¹H NMR [CDCl₃] δ 1.82-1.96(m, 3H, —CH—C and —CH₂—CH—), 2.00(s, 6H, CH₃—C═O), 4.03(d, 4H, —CH₂—O), 4.15 (2H, t), 6.2 (brs, 2H, —NH₂ brs), 8.12 (s, Ar—H H,),8.58 (s, 1H, —N—CH═N—)

### EXAMPLE-8

### This example illustrates the preparation of 9-[4-Acetoxy-3-(acetoxymethyl) butyl]-2-amino-9H purine: Formula (6)

To the mixture of 2-Amino-6-Chloropurine (20 g, 1 Eq.), methanol (400 ml.) and triethylamine (30 ml.; 1.3 Eq.) taken in round bottom flask, thiocresol (95 g, 2.6 Eq.) was added in portions after regular intervals and the reaction mixture left at room temperature with stirring for 21 hrs.

Obtained solid filtered off, washed with pet ether and dried to get 27.12 g white solid of 2-Amino-6-[(4-methylphenyl)thio]purine. Above obtained solid, K₂CO₃ (29.83 g, 2 Eq,), triethyl3-bromopropane-1,1,1-tricarboxylate (42.85 g, 1.2 Eq,) and dry DMSO (15 ml) were stirred together at 50° C. After 4 hrs. water (18 ml) was added, stirred for 10 min. and extracted with DCM. The combined organic extract were washed with brine, dried over sodium sulphate and on concentration on rota-evapour, 44.58 g product was obtained.

To a cooled (20° C.) solution of above prepared triester (44.58 g, 1 Eq,) in methanol (1040 ml), solution of sodium methoxide (6.83 g, 1.5 Eq,) in methanol (450 ml) was added with stirring at 20° C., stirring was continued for 4 hrs at same temperature. The reaction mixture was cooled to 15° C. and held at this temperature for 30 min. (till the complete product was precipitated). Solid was filtered off washed with methanol 45 ml and dried at 40° C. to get 26.96 g of 2-Amino-6-[(4-methylphenyl)thio]-9-[methyl 2-carbomethoxy butanoate-4-yl]-9H purine, which was stirred at 20° C. with sodium borohydride (9.43 g, 3.7 Eq,) and dry DCM (135 ml). Methanol (67.4 ml) was added drop wise over a period of 2 hrs. at 20° C. Then reaction mixture was left to stir for at r.t. After 6 hrs. water (135 ml) was added, followed by drop wise addition of conc. HCl to maintain pH 6.7 to 7.0 keeping the reaction temperature 20° C. Methylenechloride and methanol were removed under vacuum until a reaction volume (70 ml) was obtained. The reaction mixture was cooled to 5° C. and stirred at this temperature for 30 min. Filtration and pet-ether washing yielded 21.11 g of 2-Amino-6-[(4-methylphenyl)sulfanyl]-9-[(4-hydroxymethyl)butyl]-9H purine that was reacted with NEt₃ (4.74 g, 0.8 Eq,) and 4-dimethylaminopyridine (catalytic) in DCM (735 ml), acetic anhydride (33.6 g, 0.568 Eq,) was added drop wise over 20 to 30 min. at such rate to control the reflux. The reaction mixture was heated under reflux for further 7 hrs. The reaction mixture was cooled to 20° C. and neutralized with 20% NaOH solution to pH 6.4-6.5. The DCM layer was separated and extracted with DCM (3 times with 150 ml). The combined DCM layer evaporated and obtained syrup (18.37 g) was used for next reaction without purification.

Above obtained 9-[4-Acetoxy-3-(acetoxymethyl)butyl]-2-amino-6-[(4-methylphenyl)sulphenyl]-9H purine (18.37 g, 0.001 Mole), Raney-Nickel slurry (83.18 g) and ethanol-water (1:1 v/v; 525 ml.) were stirred together and heated under gentle reflux. After 3 hrs. the product was filtered through celite and the filtrate was evaporated under reduced pressure. Crude was purifed by flash chromatography on neutral alumina to get 10 g of pure Famciclovir.

Advantages of the present invention are:
In alkylation of intermediate of formula (2) results in desired N-9 alkylated product formula (5) in higher percentage which can be directly isolated in pure form in high yield from reaction making it easier operation.
The method for preparation of thio derivative of formula (2) is modified so as to avoid the use of reagents like trifluoroacetic anhydride, pyridine, cryogenic temperature, hydrogen peroxide which can be easily scaled up.
Desulfurisation in final step is achieved by using simple Raney Nickel instead costly palladium supported catalyst

1. Purines of general formula (1) 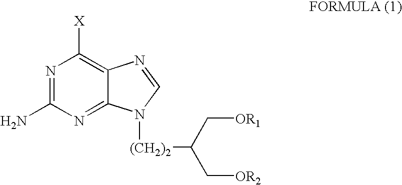
wherein X is hydrogen, thioaryl; R₁ and R₂ are hydrogen or acetyl.

2. A process for the preparation of purines of formula (1), the said process comprises the steps of;
(a) reacting an aminopurine derivative of formula (2), 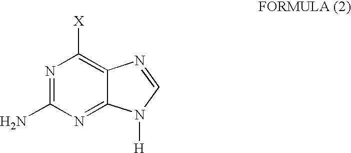
wherein X is 4-methylphenylthio, 4-chlorophenylthio with a triester of formula (4) 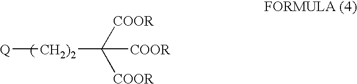
wherein Q is leaving group and R is C₁₋₆ alkyl preferably methyl or ethyl group, in presence of an organic solvent under constant agitation at about 50° C. for a period of 2 to 5 hrs. to obtain an intermediate derivative of formula (5) 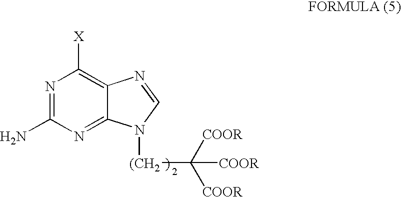
wherein X is 4-methylphenylthio, 4-chlorophenylthio and R is C₁₋₆ alkyl preferably methyl or ethyl group;
(b) cooling the reaction mixture to a temperature at about 15° C. to obtain the solid intermediate derivative of formula (5);
(f) treating the compound of formula (5) with an alkoxide base in an alcoholic solvent at ambient temperature to obtain a diester;
(g) reducing and acylating the diester in situ to obtain the intermediate compound of formula (6), and 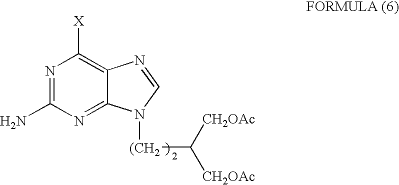
(h) desulfurising the intermediate of formula (6) with Raney nickel to obtain the compound of formula (1).

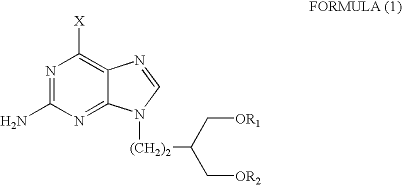

3. A process as claimed in claim 2, wherein the organic solvent for preparing compound of formula (2) and washing of compound of formula (4) is alcohol.

4. A process as claimed in claim 3, wherein the alcohols are methyl and ethyl alcohol.

5. A process as claimed in claim 2, wherein in step (c) the alkoxide base is alkoxide base of alkali metals preferably sodium alkoxide.

6. A process as claimed in claim 2, wherein preparation of 6-thioderivative is carried out by reacting 2-Amino-6-chloropurine with arylthiol in an alcoholic solvent and an organic base over a temperature range of 0° C. to boiling point of solvent preferably 25-30° C.

7. A process as claimed in claim 2, wherein the organic bases used for the preparation of 2-Amino-6-chloropurine is selected from the group comprising of triethylamine, ethyldiisopropylamine, DBU in an alcoholic solvents.

8. A process as claimed in claim 7, wherein the alcoholic solvent is selected from the group comprising of methanol, ethanol and isopropanol.
